(19) United States
(12) Patent Application Publication
(10) Pub. No.: US 2023/0364308 A1
(43) Pub. Date: Nov. 16, 2023

(54) IMPLANTABLE MEDICAL DEVICE FOR BONE REPAIR

(71) Applicants: INSTITUT POLYTECHNIQUE DE GRENOBLE, Grenoble (FR); CENTRE NATIONAL DE LA RECHERCHE SCIENTIFIQUE, Paris (FR); CENTRE HOSPITALIER UNIVERSITAIRE GRENOBLE ALPES, La Tronche (FR); CENTRE HOSPITALIER ANNECY GENEVOIS, Epagny Metz-Tessy (FR); UNIVERSITE GRENOBLE ALPES, Saint Martin D'Heres (FR)

(72) Inventors: Catherine PICART, Grenoble (FR); Georges BETTEGA, Grenoble (FR); Michael BOUYER, Sevrier (FR)

(21) Appl. No.: 18/246,166

(22) PCT Filed: Sep. 21, 2021
(86) PCT No.: PCT/EP2021/075932

§ 371 Date: Mar. 21, 2023

(30) Foreign Application Priority Data
Sep. 22, 2020 (EP) 20315416.6

(51) Int. Cl. A61L 27/56; A61L 27/34

(52) U.S. Cl. CPC A61L 27/56; A61L 27/34; A61L 2430/02

(57) ABSTRACT

An implantable medical device for bone repair following a loss of bone substance including a scaffold having a three-dimensional structure and having at least one polymer, a film having at least one protein from the Bone Morphogenetic Proteins (BMP) family, including the scaffold defines an internal volume having a three-dimensional mesh delimiting pores, the pores being open and interconnected, the largest dimension of each pore being greater than 200 µm, the scaffold having a minimum porosity of 80%, and in that the film coats the three-dimensional mesh. Also relates to the field of implantable medical devices. One application concerns the field of repairing bone following a loss of bone substance. The disclosure is applicable to large-volume bone defects ranging from 2 cm³ to 15 cm³.

### TECHNICAL FIELD OF THE INVENTION

The present invention relates to the field of implantable medical devices. One particularly advantageous application concerns the field of repairing bone following a loss of bone substance. The invention is particularly applicable to large-volume bone defects ranging from 2 cm³ to 15 cm³.

### PRIOR ART

To date, autologous bone grafts remain the primary clinical solution for treating extensive bone loss and trauma, but they suffer from several drawbacks, in particular limited availability, pain for the patient, additional healing time and donor-site morbidity.

Tissue engineering using synthetic scaffolds, bioactive factors and/or stem cells offers alternative therapeutic strategies and is promising for bone regeneration.

However, these techniques are still not suitable for the repair of large bone defects (about 5 cm³), which remains challenging. In particular, for large bone defects, a structural synthetic scaffold may not be sufficient to allow for complete regeneration.

Ceramics, in particular hydroxyapatite (HAP) and tricalcium phosphate (TCP) composites are the most biomimetic scaffolds, but are brittle and exhibit variable biodegradability. Moreover, they induce a basic level of bone formation. Metals such as titanium are interesting for their mechanical properties, however the high rigidity thereof, which is greater than that of bone, generates stresses and they are not biodegradable.

The use of polymers has grown, given their versatility, modular mechanical properties and biodegradability. To date, polycaprolactone (PCL) and polylactic acid (PLA) derivatives are the most widely used in bone tissue engineering.

Of great interest is the recent development of additive manufacturing, which allows customised 3D-architectured scaffolds to be designed and which can be adapted to the size of the defect and which are easier to implement from a regulatory perspective. Polymers are particularly well-suited to the additive manufacturing of scaffolds. They can be manufactured in filament form and be 3D-printed using several techniques, including fused deposition modelling (FDM). The 3D-architectured scaffold acts as a space filler that must be mechanically stable to allow for bone growth within the pores of the scaffold.

However, for large defect areas, a structural scaffold may not be sufficient to allow for complete regeneration.

In such cases, stem cells or exogenous factors can be added to the scaffold to enhance regeneration. The use of stem cells in combination with scaffolds appears to have potential because of their secretion of factors, but is more complicated to implement, as various steps are needed to harvest the cells from the patients, expand them in culture and finally implant them back into the patient.

As an alternative to stem cell implantation, the use of growth factors aims to recruit stem cells directly to the implantation site. To date, BMP-2 has been the most widely studied, clinically approved protein due to its ability to directly target BMP receptors at the cell surface and trigger stem cell differentiation in bone cells.

The publication by MacDonald M L et al. “Tissue integration of growth factor eluting layer by layer polyelectrolyte multilayer coated implants” Biomaterials. 32 (2011) 1446-1453 describes a ceramic-based β-TCP type, i.e. non-inert, implant covered with a film that can comprise BMP-2 in quantities in the order of 10 µg/mm³.

A previous study by Bouyer M, Guillot R, Lavaud J, Plettinx C, Olivier C, Curry V, et al. Surface delivery of tunable doses of BMP-2 from an adaptable polymeric scaffold induces volumetric bone regeneration. Biomaterials. 104 (2016) 168-81 showed that it is possible to repair a femoral bone defect with a critical size in rats of less than 2 cm³ by combining a hollow polymer tube with an osteoinductive surface coating using a polyelectrolyte film as a BMP-2 carrier. One purpose of the present invention is thus to propose an optimised implantable medical implant for the repair of a large-volume bone defect.

Other purposes, features and advantages of the present invention will appear upon reading the following description and examining the accompanying drawings. It is understood that other advantages can be incorporated therein.

### SUMMARY OF THE INVENTION

In order to achieve this objective, according to one embodiment, the invention proposes an implantable medical device for bone repair following a loss of bone substance comprising: a scaffold having a three-dimensional structure and advantageously comprising at least one polymer, a film comprising at least one protein from the Bone Morphogenetic Proteins (BMP) family, characterised in that the scaffold defines an internal volume comprising a three-dimensional mesh delimiting pores, advantageously the pores being open and interconnected, advantageously each pore having a largest dimension greater than 200 µm, preferably in the order of 2,000 µm, the scaffold has a minimum porosity of 80%, and in that the film coats the three-dimensional mesh. More specifically, the film is a polyelectrolyte film, more specifically the film comprises polyelectrolytes.

This disposition ensures homogeneous regrowth of the bone within the scaffold. Said scaffold ensures circulation of the fluids, in particular blood, which will arrive within the scaffold when it is implanted within the bone defect. The porosity of the scaffold ensures that it can be covered advantageously homogeneously by the film. Advantageously, the film perfectly takes the shape of the three-dimensional mesh of the scaffold. The three-dimensional mesh is coated by the film.

The scaffold is also advantageously mechanically strong enough to stand alone within the bone volume defect. Moreover, the 3-dimensional architecture thereof allows it to withstand mechanical stresses, particularly compressive stresses.

The porosity of the scaffold is preferably such that the scaffold can be covered homogeneously by the film. Advantageously, the film is deposited by means of an automated process. This ensures that the film perfectly takes the shape of the surface of the three-dimensional structure of the scaffold.

This optimised cooperation of the scaffold due to the 3-dimensional architecture thereof and the porosity thereof with the film ensures efficient bone repair of volume defects in bone, in particular those of critical size and large volume, for example between 2 cm³ and 15 cm³.

The use of the film as a coating allows the 3D scaffold architecture to be separated from the film which is osteoinductive.

The invention allows the porosity of the scaffold and the loading of osteoinductive factors to be independently controlled so as to modulate the release of said factors, thereby ensuring homogeneous and high-quality bone growth.

### BRIEF DESCRIPTION OF THE FIGURES

The aims, purposes, features and advantages of the invention will be better understood upon reading the detailed description given of one embodiment thereof, which is illustrated by means of the following accompanying drawings, in which:

FIG. 1 shows a 3D-printed PLA scaffold according to the invention.

FIG. 2A shows a phase-contrast macro lens imaging of a device according to the invention comprising a 3D-printed PLA scaffold coated with a film according to the invention.

FIG. 2B shows a phase-contrast confocal image of a section of a device according to FIG. 2A to visualise the film-coated filaments of the scaffold.

FIG. 2C shows a scanning electron microscope (SEM) image of the scaffold struts.

FIG. 2D shows a scanning electron microscope (SEM) image of a scratch made with a needle to assess the presence of the film on the scaffold.

FIG. 2E shows the quantification, using a µBCA assay, of BMP-2 loading in a 24-bilayer film (PLL/HA), for two crosslinking levels EDC30 and EDC70, as a function of the initial BMP-2 concentration in the loading solution.

FIG. 2F shows the quantification of BMP-2 release by a 24-bilayer film (PLL/HA) as a function of the BMP-2 concentration initially loaded, for two crosslinking levels EDC30 and EDC70.

FIGS. 3A to 3C show the kinetic quantification of the bone formation using CT images obtained with two levels of crosslinking of the film and two doses of BMP-2. The EDC30 and EDC70 films loaded with BMP-2 doses (20 and 110 µg/cm³ of scaffold) were compared. Two negative controls were added: a film-coated implant without BMP and loss of substance without complement. CT-scan scores were calculated from CT images as a function of time and corresponding exponential fits to the data for the EDC30 (FIG. 3A) and EDC70 (FIG. 3B) groups. The plateau value (Bmax), the characteristic time (T) deduced from the fits and the goodness of fit R² are given in the corresponding tables Table 2 and Table 3.

FIG. 3C shows the quantity of total bone volume as a function of time (D29, D50 and D90) and as a function of the BMP-2 dose, for the two crosslinked films EDC30 and EDC70. The linear fits are also shown.

FIG. 4 shows 3D reconstructions obtained from the CT images showing the kinetics of bone regeneration for four representative conditions: negative control (Ctrl -) (film-coated scaffold without BMP-2 in the film), film-coated scaffold with a low BMP-2 dose (BMP50, LD) and a high BMP-2 dose (BMP110, HD) and a bone graft (BG). The total length of the implant, corresponding to the distance from the lower edge of the mandible to the place of the bone substance loss, is 4 cm, direction X in the diagram.

FIGS. 5A to 5E show the quantitative analysis of the kinetics of bone formation followed by CT images for EDC30 films loaded with two BMP-2 doses. The film-coated scaffolds were loaded with BMP-2 at 50 (LD, n = 6) and 110 µg/cm³ (HD, n = 5) and the bone regenerative capacity thereof was compared to the bone autograft BG, (n = 4). FIGS. 5A to 5C show box plot data for the total bone volume (FIG. 5A), poorly mineralised (FIG. 5B) and highly mineralised (FIG. 5C) bone volumes as a function of the BMP-2 dose LD versus HD compared to BG. * p <0.05; ** p <0.01.

FIG. 5D shows CT-scan scores as a function of time and corresponding exponential fits to the data for the EDC30 films; the corresponding plateau value (Bmax), characteristic time (T) deduced from the fits and the goodness of fit R² are given in Table 4.

FIG. 5E shows the percentage of bone outside the implant (referred to as “ectopic bone”) as a function of time for LD and HD.

FIGS. 6A to 6E show the quantitative micro-CT analysis of bone formation at 3 months (D91) after explantation. FIG. 6A shows the 3D reconstructions for the negative control (ctrl-, EDC30 film-coated scaffold without BMP-2), film-coated scaffolds containing low-dose LD and high-dose HD BMP-2, and the bone graft BG.

FIG. 6B shows a box plot of the total bone volume. ** p <0.01.

FIG. 6C shows a box plot of the bone volume formed as a function of the total BMP-2 dose per implant.

FIG. 6D shows a box plot of the bone mineral density (BMD) as a function of the BMP-2 dose. * p <0.05

FIG. 6E shows a box plot of the homogeneity score measured for LD and HD. In each box in FIGS. 6B to 6E, the coefficient of variation of the data is given in %. * p <0.05; ** p <0.01.

FIGS. 7A and 7B show the histomorphometric analysis of the histological images taken after explantation.

FIG. 7A shows a box plot of the quantity of bone present in a histological image (BA) over the total area of the image (TA).

FIG. 7B shows a box plot of the percentage of bone within each histological image of the implant (S1, S2, S3).

The drawings are provided by way of example and are not intended to limit the scope of the invention. They are representations intended to simplify the understanding of the invention.

### DETAILED DESCRIPTION OF THE INVENTION

Before giving a detailed review of embodiments of the invention, optional features are set out below, which can be used as an alternative to or in combination with one another:

According to one example, the scaffold is inert, and also referred to as bioinert. It does not have the ability to induce bone regrowth, i.e. it is not osteoinductive. The scaffold acts as a support for the film and as a passive support for bone regrowth. The scaffold has an osteoconductive role in guiding bone regrowth.

According to one example, the scaffold comprises at least one polymer selected from the group consisting of polylactic acid (PLA), polycaprolactone (PCL), polyglycolic acid (PGA) or copolymers thereof. Preferably, the scaffold does not comprise ceramics, in particular such as β-TCP (beta-tricalcium phosphate).

According to one example, the three-dimensional mesh has orientation angles between 0° and 120°, preferably -120°/+120°, preferably the three-dimensional mesh has a pore orientation of -45°/+45° C.

According to one example, the film comprises a quantity of BMPs ranging from 0.01 mg/cm³ to 0.2 mg/cm³, more precisely from 0.01 mg/cm³ to 0.15 mg/cm³ or even from 0.01 mg/cm³ to 0.12 mg/cm³, for example from 0.017 mg/cm³ to 0.072 mg/cm³.

Preferably, the BMP is BMP-2. This selection of the BMP dose is of particular interest, as it is much lower than the quantities of the prior art, which is surprising given the idea that a higher dose would be beneficial for bone repair. The selected dose of BMPa has surprisingly allowed possible side effects to be avoided, such as inflammation around the implant and the production of bone outside the implant (ectopic bone). This significantly lower dose of BMP compared to the prior art is made possible by the specific features of the scaffold, such as the porosity and pore size thereof.

According to one example, the film is a polyelectrolyte film, more specifically the film comprises polyelectrolytes.

According to one example, the film is a crosslinked multilayer polyelectrolyte film.

According to one example, the film is crosslinked with a crosslinking agent, preferably 1-ethyl-3-(3-dimethylaminopropyl) carbodiimide hydrochloride (EDC), preferably at a concentration of between 30 mg/mL (EDC30) and 70 mg/ml (EDC70).

According to one example, the crosslinked film has a concentration of between 30 mg/mL and 70 mg/mL of 1-ethyl-3-(3-dimethylaminopropyl)carbodiimide hydrochloride (EDC). The crosslinking guarantees the formation of covalent bonds within the film. According to one example, the device is adapted for bone repair of a volume between 2 cm³ and 15 cm³, preferably greater than 6 cm³, more preferably greater than 10 cm³.

According to one aspect, the invention relates to a method for manufacturing an implantable medical device as described hereinabove, characterised in that the scaffold is manufactured by 3D printing.

According to one example, the manufacturing method comprises a step of sterilising the implantable medical device.

The implantable medical device according to the invention is intended for bone repair.

The medical device according to the invention is intended to be implanted in the human or animal body, more specifically in a bone defect. In particular, the device is particularly effective when the bone defect is a volume defect. According to one possibility, the defect is of critical size. The term ‘critical’ is understood to mean that the bone defect cannot be filled spontaneously by the usual healing mechanisms alone. Advantageously, the device according to the invention is particularly adapted for large bone defects. The term ‘large bone defect’ is understood to mean that the bone volume defect is greater than 2 cm³, more specifically greater than 6 cm³, more preferably greater than 10 cm³ and advantageously up to 15 cm³.

The implantable device according to the invention comprises a scaffold and a film covering said scaffold and advantageously comprising a protein of the Bone Morphogenetic Protein (BMP) family. The implantable medical device according to the invention is also referred to as an activated device, a bioactive medical device or an active or bioactive implant when it comprises BMPs.

The scaffold has a three-dimensional structure advantageously intended to be inserted into the bone defect to be repaired. The scaffold is intended to fill at least part of the bone defect, and preferably all of it.

The term ‘three-dimensional structure’ is understood to mean that the scaffold has a three-dimensional organisation, also referred to as 3-dimensional conformation. The 3-dimensional conformation goes beyond the definition of the scaffold perimeter and also defines the structure of the internal volume of the scaffold.

The scaffold is advantageously shaped to match the bone defect to be repaired. The scaffold further provides support for the film and acts as a guide for bone regrowth.

The scaffold is a solid. It is a geometric shape with 3 dimensions: height (Z), width (X), and depth (Y). According to one possibility, the aspect ratio, i.e. the ratio of one of the dimensions to the other, is less than 100.

By way of example, the scaffold is a plaque with a width (X) of 4 cm, a height (Z) of 3 cm and a depth (Y) of 1 cm, i.e. a volume of 12 cm³.

The scaffold defines an internal volume. The scaffold comprises a mesh delimiting pores. Advantageously, the mesh extends throughout the volume of the scaffold. The mesh extends within the scaffold into the internal volume. The mesh is three-dimensional. It extends into the internal volume along the three dimensions of the scaffold.

Advantageously, the scaffold is said to be porous. Preferably, the scaffold has a porosity of at least 80%, preferably in the order of 85%, more preferably in the order of 90%. This porosity is in particular due to the mesh.

The porosity of the scaffold is understood as the empty volume of the internal volume of the scaffold.

This high porosity of the scaffold ensures optimal colonisation of the scaffold by, for example, progenitor cells and then by bone over time, while also providing mechanical support.

The scaffold comprises pores which are advantageously open, i.e. the pores are connected to one another. In such a case, the pores form channels. The pores are thus also referred to as being interconnected. This type of porosity allows fluids to circulate within the scaffold.

Preferably, the pores of the scaffold have at least one dimension greater than 200 µm, preferably in the order of 2,000 µm, plus or minus 10%. In a preferable manner, the dimension is the largest dimension of the pore. According to one possibility, the largest dimension of the pore lies in a plane parallel to the plane in which lie the filaments or trabeculae forming the mesh described hereinbelow.

The scaffold according to the invention provides a temporary mechanical support for bone repair while the bone forms and grows within the pores.

According to one possibility, the scaffold is biodegradable. According to one possibility, the scaffold is bioresorbable.

The scaffold advantageously has interesting mechanical properties. The scaffold is referred to as a polymer scaffold.

Preferably, the scaffold is architectured in 3 dimensions, for example by 3D printing, in particular using fused deposition modelling (FDM). 3D-printed scaffolds are particularly adapted for repairing large bone defects. The scaffold can be adapted to the geometry of the bone defect to be filled, even if this geometry is complex. The term ‘the scaffold is architectured’ is understood to mean that it is manufactured, built or arranged as a whole organised so as to give it a defined architectural character.

For example, the mesh of the scaffold is formed by trabeculae or filaments. The filaments or trabeculae are disposed in different orientations, in particular to control porosity. The orientation can be 0°/90°, 0°/15°, 0°/30°, 0°/45°, 0°/60° or -15°/+15°, -30°/+30°, -45°/+45°, -60°/+60°, 60°/120°. The disposition of the intersecting filaments or trabeculae defines struts and advantageously pores.

The filaments or trabeculae form the mesh of the scaffold. For example, the filaments or trabeculae are made of an inert material such as PLA, which advantageously has a diameter of 400 µm in the scaffold. Preferably, the filaments are spaced apart, thereby defining a filament spacing, i.e. a strut of between 200 µm and 2.5 mm, more specifically between 1 and 2.5 mm.

The scaffold is particularly adapted to being covered by a film, preferably a polyelectrolyte film, preferably a polyelectrolyte multilayer film.

Polyelectrolyte multilayer films are formed by condensation-crosslinking complementary groups located on the adjacent layer. The European patent document No. 1 535 952 describes a practical method for producing crosslinked polyelectrolyte multilayer films.

The European patent document No. 1 535 952 describes a crosslinked polyelectrolyte multilayer film, the method of manufacture whereof comprises reacting layer pairs of an anionic polymer with carboxylic groups and of a cationic polymer with amino groups in the presence of a carbodiimide coupling agent.

The polyelectrolyte film is more preferably biocompatible. In particular, such biocompatible films can make any coated surface biocompatible. As a result, such biocompatible materials when applied to biological tissues, in particular inside the body, have the advantage of not irritating the surrounding tissues, of not causing an abnormal inflammatory response and of not causing an allergic or immunological reaction.

The polyelectrolyte film advantageously comprises two or more layers, the film is thus referred to as a polyelectrolyte multilayer film.

Preferably, each further layer has the opposite charge of the previous layer. The architecture of the film is precisely designed and can be controlled to an accuracy of 1 nm with a range of 1 to 50,000 nm, preferably 100 nm to 30 µm, and with precise knowledge of the molecular composition thereof.

The number of layer pairs in a prepared polyelectrolyte multilayer film can vary over a wide range and depends on the desired thickness. In particular, the number of layer pairs can vary from 5 to 2,000, preferably from 5 to 1,000, more preferably from 5 to 100, preferentially from 20 to 30, preferentially 24.

As mentioned hereinabove, the thickness of the film can generally vary from 1 nm to 50,000 nm. A film is considered to be thick when the thickness thereof is greater than 300 nm. According to the invention and in one specific embodiment, the thickness of the film ranges from 500 nm to 20 µm, more preferably from 1 to 10 µm.

In one specific aspect of the invention, the cationic polymer of the film comprising an amino group is poly-L-lysine (or PLL).

In one specific aspect of the invention, the anionic polymer comprising an amino group is hyaluronic acid or a salt thereof, such as sodium hyaluronan (also typically referred to as HA), or a mixture thereof.

The polyelectrolyte multilayer film is more preferably a PLL/HA film.

The polyelectrolyte multilayer film can advantageously be loaded with a protein such as an osteoinductive factor. A loaded film is of particular interest as it acts as a biomimetic reservoir for protein storage and release. For example, the controlled delivery of growth factor from a biomaterial surface can be achieved under very satisfactory conditions, as it allows the growth factor to be conveniently concentrated and released locally, and protected from degradation by enzymes in tissue fluids, in particular proteases.

The proteins can be incorporated by adsorption or diffusion or by coupling said materials to at least one of the polyelectrolytes and adsorption of said polyelectrolyte.

The film forms a coating on the scaffold. Preferably, the film coats the mesh. The mesh is covered with the film. The film will thus cover the mesh, more specifically, the struts of the mesh are covered. Preferably, the entire mesh, and thus the entire scaffold, is covered, and thus each strut is covered. The mesh is said to be film-coated. The film covers the mesh in a homogeneous manner.

The film advantageously comprises osteoinductive factors and preferably proteins of the bone morphogenetic protein (BMP) family. In this respect, the film plays a biomimetic role by delivering BMPs and promoting bone regrowth. Biomimetics is the application of knowledge from biological models. The film loaded with BMPs advantageously has an osteoinductive action. The homogeneous presence of the film on the mesh ensures homogeneous bone regrowth.

In a preferred example, the osteoinductive factors are proteins of the Bone Morphogenetic Protein (BMP) family, more preferably BMP-2, BMP-7, BMP-4, BMP-9, BMP-6 or a mixture of BMPs or a BMP heterodimer or fragments of BMPs or peptides derived from these BMPs.

The following description refers to BMPs, but also applies to other osteoinductive factors.

The film forms a reservoir for BMPs, advantageously corresponding to a biomimetic reservoir. The BMPs are advantageously adsorbed in the film in a non-covalent manner, which allows for in vivo release after implantation of the device.

The quantity of BMP in the film is advantageously selected such that the quantity of BMP in the device is between 0.01 mg/cm³and 0.2 mg/cm³, more specifically between 0.017 mg/cm³ and 0.072 mg/cm³, more specifically between 0.02 mg/cm³ and 0.08 mg/cm³. By way of example, the quantity of BMP in a 12 cm³ bone defect is between 240 µg and 1,000 µg. The mass of BMP is given per unit volume of the scaffold. The quantity of BMP is thus reduced by 1/20 to 1/75 compared to commercially-available collagen sponges.

Advantageously, the device according to the invention allows the spatial delivery of the BMPs to be controlled. The delivery of the BMPs is perfectly controlled via the 3D architecture, i.e. the mesh of the scaffold covered by the film. This controlled spatial delivery ensures spatially localised bone regrowth within the volume of the scaffold. Bone regrowth in the bone defect to be repaired is very advantageously homogeneous.

Advantageously, this selection of the quantity of BMP ensures the absence of local inflammation, swelling, bone pseudocyst or bone resorption. Similarly, the quantity of BMP limits ossification outside the implant.

The film is advantageously loaded with BMPs by soaking the scaffold in a solution with a defined concentration of BMPs.

According to one possibility, the quantity of BMPs in the film can be modulated according to the concentration of BMPs in the solution in which the entire scaffold is soaked.

According to one possibility, the quantity of BMPs in the film can be modulated according to the physical-chemical properties of the film, in particular the thickness and degree of crosslinking thereof.

According to one embodiment, the film is a multilayer polyelectrolyte film. Advantageously, the film is formed by alternating layers of poly-L-Lysine (PLL) and hyaluronic acid (HA). The film is formed by a stack of layer pairs, each pair comprising polymers of poly-L-Lysine (PLL) and hyaluronic acid (HA). A pair is also referred to as a bilayer. For example, there are 24 bilayers.

According to one embodiment, the film is crosslinked before loading with osteoinductive factors.

The implantable medical device according to the invention can advantageously be sterilised according to the requirements in force for this type of device, while preserving the properties and structure thereof. Preferably, the activated implantable medical device is sterilised while retaining the properties and structure thereof. Sterilisation can be carried out by gamma irradiation for example.

According to one aspect, the invention relates to a method for manufacturing an implantable medical device as described hereinabove, comprising a scaffold architecting step. Architecting corresponds to the manufacturing of the scaffold. The scaffold is manufactured by 3-dimensional printing. Manufacturing by polymer printing allows for an implant whose geometry (size, shape, porosity) is controlled in 3 dimensions.

According to one example, the manufacturing step comprises depositing PLA filaments, for example of about 400 µm in diameter in a predefined pattern such as, for example, -45°/45° with a height of 200 µm and a spacing distance of ± 2 mm.

According to one example, the film is manufactured according to example 1. The film is manufactured by a step of successively depositing layers, for example made of PLL and HA, on the scaffold.

Preferably, the step of manufacturing the film comprises a crosslinking step and a step of loading the film with osteoinductive factors. According to one example, the crosslinking step is carried out before the step of loading the film with osteoinductive factors.

According to one possibility, the manufacturing method comprises a step of sterilising the implantable medical device obtained.

### EXAMPLES

### Example 1: Preparation of PLA Scaffolds, Polyelectrolyte Multilayer (PEM) Film Coating

Parallelepipedal, advantageously biodegradable and bioresorbable, 10 × 30 × 40 mm scaffolds made of clinical-grade polylactic acid (Poly-Med, Inc, Lactoprene® 100 M Monofilament 1.75 mm) were manufactured by fused deposition modelling (3DXP - One). FIG. 1 shows a device according to the invention. PLA filaments of about 400 µm in diameter were deposited in a -45°/45° pattern with a height of 200 µm and a spacing distance of ± 2 mm. The scaffold had a porosity of 85% with fully interconnected pores. After manufacture and before coating with polyelectrolyte multilayer films, the scaffolds were stored away from moisture in a desiccator with a silica gel.

The polyelectrolyte multilayer films were deposited using 0.5 mg/mL poly-L-lysine (PLL, Sigma, France) and 1 mg/mL hyaluronic acid (HA, Lifecore, USA) and the DR3 dip-coating robot (Kirstein and Riegler GmbH) after deposition by hand of 5 mg/mL polyethyleneimine (Aldrich). The degree of film crosslinking was controlled by incubating the coated scaffolds in 30 or 70 mg/ml of 1-ethyl-3-(3-dimethylaminopropyl) carbodiimide (EDC, Sigma, France). After UV sterilisation of the implants, BMP-2 (InductOs, Medtronic) was post-loaded in the polyelectrolyte multilayer films at initial BMP-2 concentrations in the loading solution of 17, 46 or 92 µg/mL as previously described in the publications by Bouyer M, Guillot R, Lavaud J, Plettinx C, Olivier C, Curry V, et al. Surface delivery of tunable doses of BMP-2 from an adaptable polymeric scaffold induces volumetric bone regeneration. Biomaterials. 104 (2016) 168-81 and Crouzier T, Ren K, Nicolas C, Roy C, Picart C. Layer-by-Layer films as a biomimetic reservoir for rhBMP-2 delivery: controlled differentiation of myoblasts to osteoblasts. Small. 5 (2009) 598-608. Finally, the osteoinductive coated scaffolds were rinsed, dried and stored away from moisture in a desiccator with a silica gel until implantation.

### Example 2: Characterisation of Polyelectrolyte Multilayer Films and Quantification of the Quantity of BMP-2 Loaded.

Fluorescence microscopy and scanning electron microscopy were used to characterise the film coating on the scaffold. The air-dried, polyelectrolyte multilayer films coated on the PLA scaffolds were imaged by scanning electron microscopy (SEM) using an FEI-Quanta 250 SEM-FEG under high vacuum at 15 keV using the Everhart-Thornley detector according to the publications by Bouyer M, Guillot R, Lavaud J, Plettinx C, Olivier C, Curry V, et al. Surface delivery of tunable doses of BMP-2 from an adaptable polymeric scaffold induces volumetric bone regeneration. Biomaterials. 104 (2016) 168-81 and Crouzier T, Ren K, Nicolas C, Roy C, Picart C. Layer-by-Layer films as a biomimetic reservoir for rhBMP-2 delivery: controlled differentiation of myoblasts to osteoblasts. Small. 5 (2009) 598-608. For fluorescence observations, the film-coated scaffolds were labelled with PLL^{FITC} according to the publication by Crouzier T, Sailhan F, Becquart P, Guillot R, Logeart-Avramoglou D, Picart C. The performance of BMP-2 loaded TCP/HAP porous ceramics with a polyelectrolyte multilayer film coating. Biomaterials. 32 (2011) 7543-54. They were imaged using a Leica Macrofluo fluorescence system (Z16 Apo) using a 0.8X objective and a Zeiss LSM 700 confocal microscope with a 10X objective.

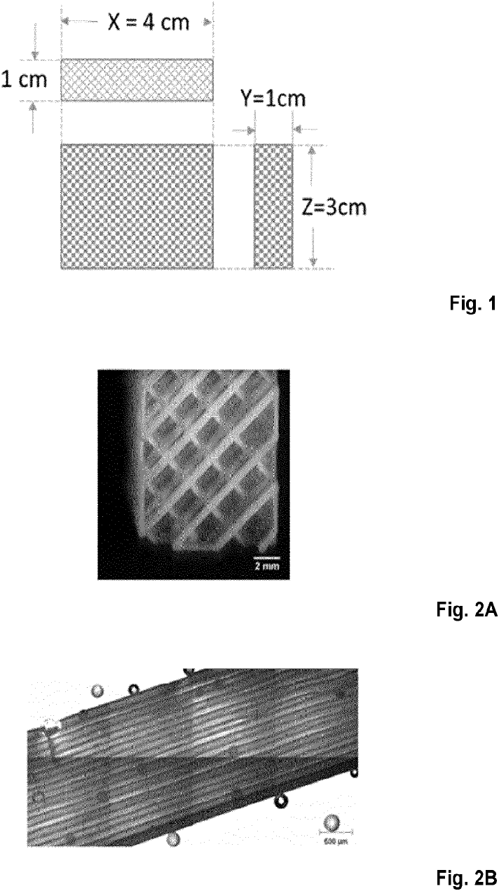

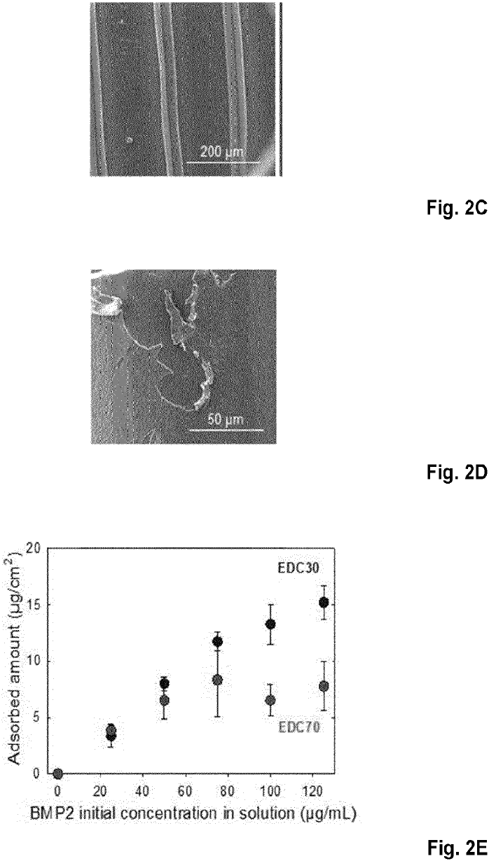

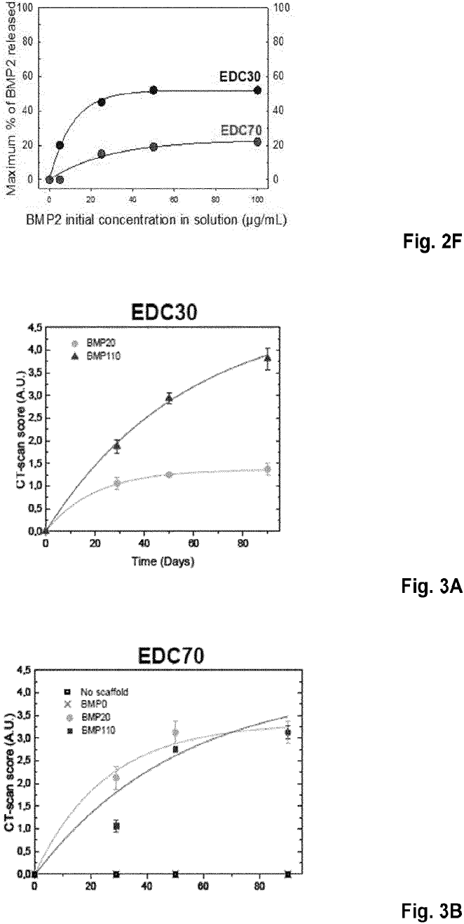

The results obtained are shown in FIGS. 2A to 2D and show a homogeneous coating of the scaffold. The mesh of the scaffold is coated with the film. The quantity of BMP-2 increased with the initial concentration of BMP-2 in the loading solution, but it reached a plateau more quickly for the EDC70 film (FIG. 2E). The quantity of BMP-2 released was higher for the EDC30 film, with the maximum percentage of BMP-2 released from the film being in the order of 50% for the EDC30 films compared to about 20% for the EDC70 films (FIG. 2F).

The quantification of BMP-2 initially loaded in the polyelectrolyte film was carried out using a micro bicinchoninic acid (µBCA) assay and the percentage of in-vitro release after several washes with physiological buffer (HEPES-NaCl), was determined by fluorescence spectrometry using BMP-2^{CF}. The results obtained can be seen in FIGS. 2E and 2F. To quantify the BMP-2 loaded in film-coated scaffolds, a µBCA assay was used for a low BMP-2 dose (BMP20), whereas the Nanodrop (Thermofisher) was used for high concentrations. The concentration of BMP-2 in the loading solution was measured initially and then after incubation with the film-coated scaffold. The quantity loaded corresponds to the difference between these two values. It is also expressed as µg of protein per volume of scaffold (µg/cm³).

Table 1. Quantification of the quantity of BMP-2 loaded in the film-coated architectural scaffold. The total volume of the scaffold was 12 cm³ and the estimated surface area from the design was 144 cm². We targeted a BMP-2 dose (unit mass per unit of scaffold volume in µg/cm³). The total quantity of BMP-2 loaded was calculated for each implant and reported in “mass per volume of implant” (µg/cm³).

TABLE 1

| | Targeted BMP-2 dose (µg/cm³) | Total BMP-2 dose loaded (µg/implant) | Volumetric dose of BMP-2 loaded (µg/cm³) |
|---|---|---|---|
| Low dose (n = 2) | 20 | 240 µg | 20 |
| High dose (n = 2) | 110 | 870 µg | 72.5 |
| Low dose (n =6) | 50 | 326 ± 80 µg | 27 |
| High dose (n = 5) | 110 | 1,000 ± 64 | 83 |

### Example 3: Repair of a Critical-Size Mandibular Defect in Vivo in a Mini-Pig

In order to optimise bone regeneration, a preliminary experiment was carried out on the mini-pig mandibles. 4 different conditions (n = 1), corresponding to two film crosslinking levels (EDC30 and EDC70) and two BMP-2 doses (BMP20 and BMP110) were initially screened. These doses expressed in µg of BMP-2 per cm³ of scaffold are the targeted BMP-2 “volumetric” doses. Knowing the surface area of the scaffold (144 cm²) and the quantity of BMP-2 loaded in the polyelectrolyte film, the concentration of BMP-2 in the loading solution (in µg/mL) in which the scaffold was soaked was defined. The quantities of BMP-2 that were effectively loaded in the film-coated 3D scaffolds were also quantified (Table 1). Two negative controls were added: an empty defect without any implant and a defect with the EDC70 crosslinked film-coated implant, but without BMP-2.

The animals were in good health. There was no postoperative infection, implant failure or evidence of a blood disorder. All of the surgical procedures were uneventful and there were no surgical complications. For one implant, the anterior edge of the bone defect had to be recut to improve the fit with the implant. All of the titanium plates were stable and fixed to the host bone (no loosening or loss of adhesion of the screws). During explantation, it was impossible to macroscopically identify the active (BMP-loaded) implant of the host bone from bone reconstruction or scar tissue. Complete blood count, haptoglobin and protein electrophoresis were measured to assess inflammation and haemostasis. Aspartate aminotransferases (ASAT), alanine aminotransferases (ALAT), alkaline phosphatase (ALP), gamma-glutamine transferase (GGT) and bilirubin were measured to assess liver function. Serum creatinine and urea were measured to assess kidney function. The analysis of the blood samples did not reveal any abnormalities. It was concluded that the scaffold with or without the film and/or BMP-2 did not cause general inflammation, nor did it cause any particular liver or kidney reaction due to this experiment, regardless of the condition.

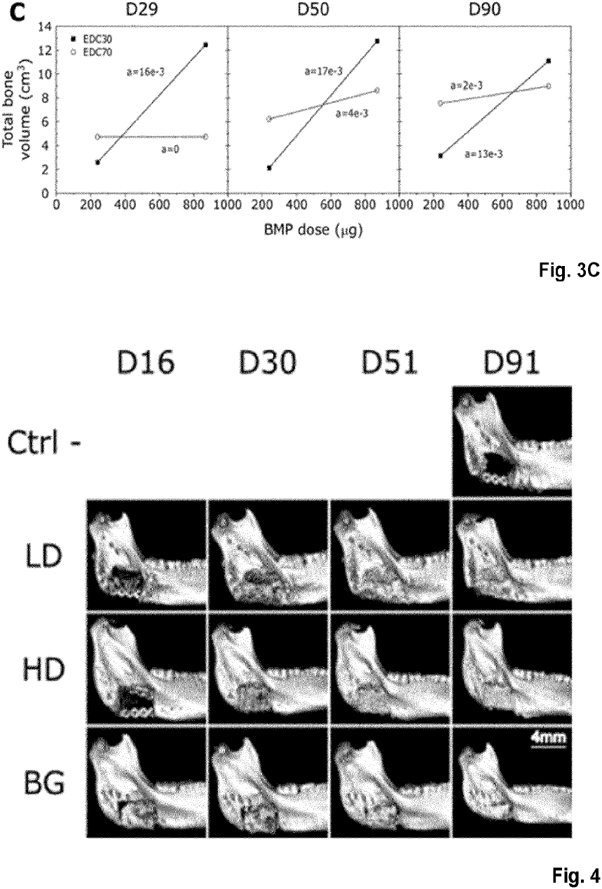

CT images were acquired during the monitoring period. For each acquisition, a CT-scan score was blinded by four clinicians. The results are shown in FIGS. 3A and 3B. Negative controls did not show bone formation throughout the monitoring period. All groups with BMP-2 exhibited bone regeneration, irrespective of the degree of crosslinking of the film and the BMP-2 concentration.

The CT-scan score was used to calculate a plateau value (Bmax) and a characteristic plateau time (T) by fitting an exponential function to the experimental data.

TABLE 2

| BMP (µg/cm3) | Bₘₐₓ (A.U.) | T (Days) | R² |
|---|---|---|---|
| 20 | 1.4 ± 0.0 | 21 ± 2 | 0.999 |
| 110 | 4.9 ± 0.7 | 60 ± 14 | 0.999 |

For the EDC30 films, for which the calculated values are shown in Table 2 hereinabove, the scores increased steadily before reaching a plateau value Bmax, which was higher for BMP110 than for BMP20 (4.9 ± 0.7 versus 1.4 ± 0.0 respectively). The time to reach the plateau (τ) was about 3 times faster for the low dose than for the high dose (21 ± 2 versus 60 ± 14 days).

TABLE 3

| BMP (µg/cm3) | Bₘₐₓ (A.U.) | T (Days) | R² |
|---|---|---|---|
| 20 | 3.3 ± 0.4 | 30 ± 12 | 0.995 |
| 110 | 4.2 ± 3.0 | 88 ± 111 | 0.971 |

In contrast, for the EDC70 films, for which the calculated values are shown in Table 3 hereinabove, the exponential fit to the data was poor for the highest BMP-2 concentration, and there was no clear dose-dependence. For the low dose, Bmax was 3.3 ± 0.4 and τ was 30 ± 12 days.

The quantification of the total regenerated bone volumes from the CT images for the different time points (D29, D50, D90) confirmed the dependence of bone repair on the BMP-2 dose for the EDC30 films but not for the EDC70 films. The results are shown in FIG. 3C. The quantity of poorly mineralised bone and of highly mineralised bone was also plotted. The slopes of the linear fits were higher for the highly mineralised part, suggesting that this type of bone has more influence on the total bone volume than the poorly mineralised one.

After 3 months, µCT images were acquired after explantation of the scaffolds and used to calculate the total bone volume (BV) and the bone mineral density (BMD) for all samples. BV also exhibited a clear dependence on the BMP-2 dose, independently of the level of crosslinking, whereas BMD was rather stable. µCT imaging of the EDC70 films confirmed that there was no visible BMP-2 dose dependence for this film condition.

Overall, these data established the critical size of the mandibular bone defect, the difference in bone repair kinetics as a function of BMP-2 dose and the influence of the level of film crosslinking on the quantity of newly-formed bone. A clear BMP-2 dose dependence was observed for the EDC30 films.

### Example 4: The Influence of the BMP-2 Dose on Bone Repair Kinetics and The Quantity of Highly Mineralised Bone.

Experiments were repeated with more mini-pigs per condition to quantitatively assess the effect of the BMP-2 doses. We focused only on the EDC30 films and selected two BMP-2 doses. The highest dose BMP110 was kept and the low dose of BMP-2 was increased to BMP50 (n = 5 for BMP110 and n = 6 for BMP50). A bone graft was added as a positive control (n = 4) and a film-coated scaffold as a negative control (EDC30 film without BMP-2). Moreover, an additional earlier time point (D16) was added for the CT-scan acquisitions.

Once again, there were no surgical complications. In two cases, the bone graft was in two pieces due to the small size of the iliac bone (donor site), but in all cases the defect was completely filled. In three cases (two cases of bone grafting and one case of a high BMP-2 dose) a small serous collection was found around the implant, and in one case a suppurated collection with a cutaneous fistula appeared at D86.

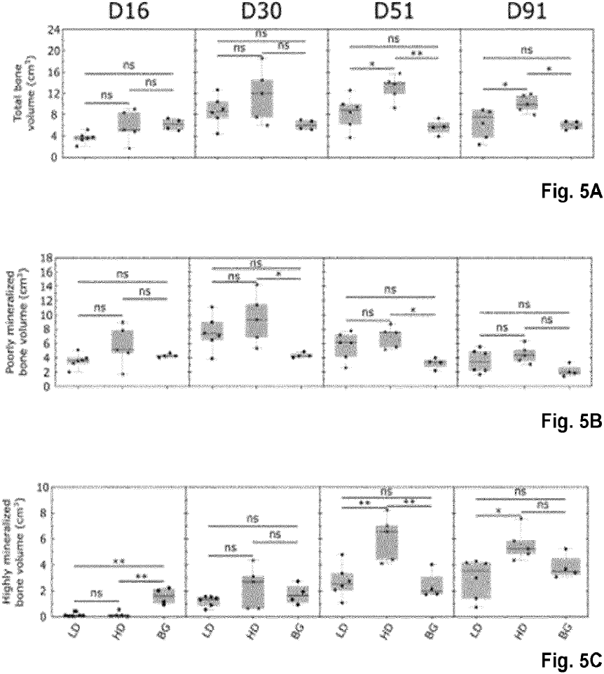

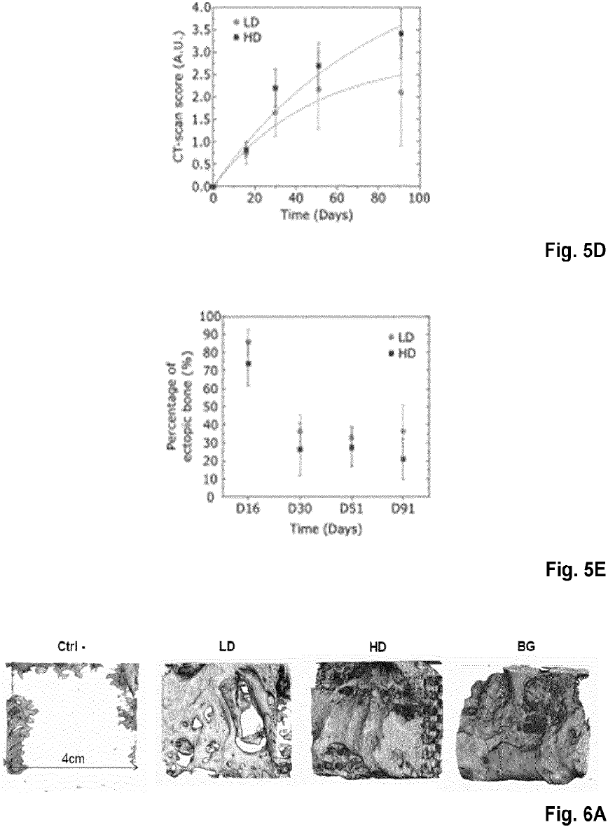

FIG. 4 shows three-dimensional scan images over time for a bone autograft, synthetic implants with the two BMP-2 doses and the negative control. At first glance, it was observed that the bone autograft mineralised over time, but that the total quantity thereof did not change. In contrast, the film-coated and BMP-2 loaded scaffolds induced bone repair over time and mineralisation was also visible. The total bone volume, the poorly mineralised and the highly mineralised bone volumes were quantified from the CT images (FIGS. 5A, B, C). The total bone volume increased over time to reach a plateau, with a significantly higher increase for the high BMP-2 dose condition than for the low dose (FIG. 5A). The quantity of bone graft volume remained constant. The quantity of poorly mineralised bone quickly increased over time for the implants loaded with low and high doses of BMP-2, but with no statistical difference between the two doses (FIG. 5B). In contrast, the quantity of highly mineralised bone increased as a function of time, reaching a peak at D51 (FIG. 5C). It was significantly higher for the high dose than for the low dose of BMP-2.

The CT-scan scores were plotted against time and the data fitted with an exponential function (FIG. 5D). The score increased for the two BMP-2 doses, but again with different characteristics: Bmax was lower for the low BMP-2 dose scaffold than for the high BMP-2 dose scaffold (2.9 ± 1.2 versus 5.2 ± 2.5). T was about two times lower for the low BMP-2 dose (71 ±40 days) than for the high BMP-2 dose (142 ± 96 days).

TABLE 4

| BMP (µg/implant) | Bₘₐₓ (A.U.) | T (Days) | R² |
|---|---|---|---|
| 326 | 2.9 ± 1.2 | 71 ±40 | 0.978 |
| 1000 | 5.2 ± 2.5 | 142 ± 96 | 0.977 |

The part of the regenerated bone that was forming outside the implant (FIG. 5E), which is referred to here as “ectopic bone”, was then analysed. Initially high, the fraction of bone growing outside the implant quickly reached a plateau value at around 28-35%, independently of the BMP-2 dose. The dispersion of the values was noted to be slightly higher for the high BMP-2 dose, and also slightly higher at the D91 endpoint.

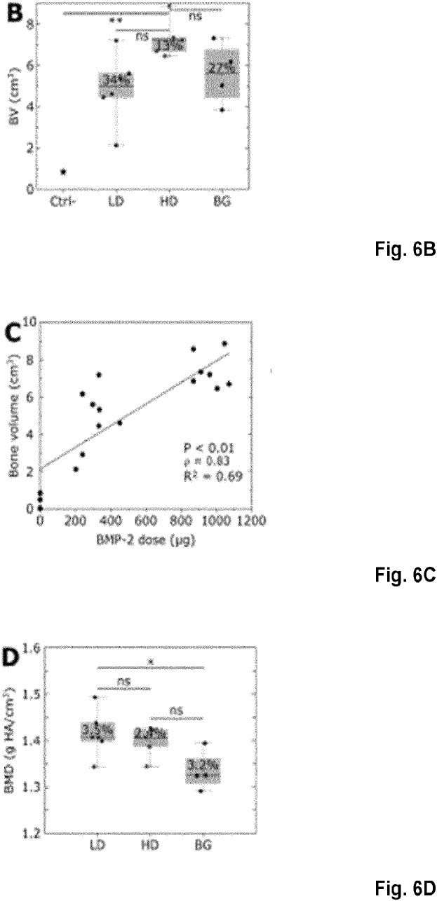

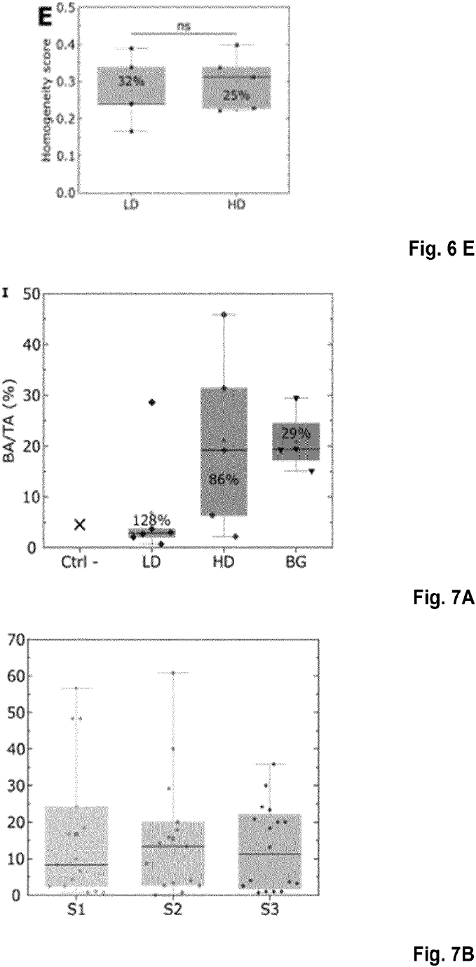

The µCT analysis performed at day 90 after the sacrifice of the mini-pigs was used to analyse the newly-formed bone in more detail. The negative control confirms the critical size of the mandibular bone defect and the bone grafts provide a positive reference value. For the low BMP-2 dose, bone formation was scarce. The quantity of bone gradually increased and the newly-formed bone entirely filled the pores of the scaffold in a homogeneous manner. There was no evidence of excessive ectopic bone formation, even at the highest doses (FIG. 6A). Bone volume was significantly higher for the high BMP-2 dose (mean value of 7.3 cm³) than for the low BMP-2 dose (mean value of 4.9 cm³) (FIG. 6B) and was also higher than for the reference bone graft. Moreover, bone regeneration was more dispersed with a low dose, with a coefficient of variation of 34% compared to 13% for the high dose. When plotting all newly-formed bone volumes against the BMP-2 dose per implant, a linear correlation was found (FIG. 6C). The BMD was not significantly different for the different conditions (FIG. 6D) and was very homogeneous with less than 3.5% variation for each experimental condition. Furthermore, the quantity of bone that developed outside the implant was not dependent on the BMP-2 dose. Finally, the homogeneity score of the bone within the scaffold was similar for the low and high doses of BMP-2 (FIG. 6E).

Overall, these data show that bone formation within the 3D-architectured scaffold is homogeneous, and that there is bone formation that is significantly dependent on the BMP-2 dose. BMP-2 mainly influences the formation of mineralised bone and does not induce ectopic bone formation.

### Example 5: Bone Homogeneity Score

The implant of defined dimensions (3 cm × 4 cm × 1 cm, total volume TV of 12 cm³) was taken as the region of interest (ROI). It was separated into ten slices of equal thickness along each axis (X, Y, Z). For each slice, the bone volume ratio (BVr = BV/TV) was calculated as the bone volume into one slice (BVs) divided by the volume of the slice of interest (corresponding to TV/10). This quantification was carried out for each axis: the standard deviation (SD) of BVr was calculated and the homogeneity score was defined as the sum of the three SDs over the X, Y and Z axes.

A histological examination revealed the presence of mature bone with a characteristic Haversian structure in the BMP-2 loaded implants. The interface between the host bone (HB) and the newly-formed bone (NB) was visible. Imaging under unpolarised and polarised light allowed for better visualisation of the Haversian canals and the connections between osteocytes. Moreover, the interface between the host bone and the newly-formed bone was visible, as the host bone had a more lamellar structure than the newly-formed bone. Some bridges were visible between the two types of bone, which may help increase the mechanical strength of the newly-formed bone. In some cases, in particular for HD (high dose), the difference between the host bone and new bone was not even visible. In the case of bone autograft (BG), the host bone and the grafted bone were in direct contact or separated by mesenchymal tissue. In the absence of BMP-2, only mesenchymal tissue (m) was formed. With a low dose (LD) of BMP-2, the quantity of new bone was low and the mesenchymal tissue was visible. The quantity of bone per histological image (BA/TA in %) was quantified on the basis of these images (FIG. 7A). In agreement with the CT and µCT quantifications, more bone was formed for the HD, whose median value was similar to that of BG. Finally, the homogeneity of the newly-formed bone within the 3D architecture was quantified (FIG. 7B). Again, bone formation was similar for all three histological images of each sample, demonstrating the homogeneity of bone formation.

In the present application, by way of example, the statistical analyses were carried out as described hereinbelow.

OriginPro (OriginLab), Excel (Microsoft Office) and R for Mac OS X (R Foundation for Statistical Computing, CRAN) were used for all analyses. Data were expressed as mean ± standard deviation. Non-parametric data were presented by median and interquartile range. Differences between groups were assessed by analysis of variance (ANOVA) and Bonferroni post-hoc analysis or Student’s t-test. The differences between groups at p <0.05 (*) and p <0.01 (**) were considered to be significant.

The invention is not limited to the aforementioned embodiments, and includes all the embodiments covered by the claims.

1. An implantable medical device for bone repair following a loss of bone substance comprising:
a scaffold having a three-dimensional structure and comprising at least one polymer,
a film comprising at least one protein from the Bone Morphogenetic Proteins (BMP) family,
wherein the scaffold defines an internal volume comprising a three-dimensional mesh delimiting pores, the pores being open and interconnected, the largest dimension of each pore being greater than 200 µm, the scaffold having a minimum porosity of 80%,
and the film is a film comprising polyelectrolytes and coats the three-dimensional mesh.

2. The device according to claim 1, wherein the scaffold is inert.

3. The device according to claim 1, wherein the scaffold comprises at least one polymer selected from the group consisting of polylactic acid (PLA), polyglycolic acid (PGA), polycaprolactone (PCL), or copolymers thereof.

4. The device according to claim 3, wherein the three-dimensional mesh has orientation angles of between -120° and +120°.

5. The device according to claim 1, comprising a quantity of BMPs in the range 0.01 mg/cm³ to 0.2 mg/cm³.

6. The device according to claim 1, comprising a quantity of BMPs of between 0.017 mg/cm³ and 0.072 mg/cm³.

7. The device according to claim 1, wherein the film is a crosslinked multilayer polyelectrolyte film.

8. The device according to claim 1, wherein the crosslinked film has a concentration of between 30 mg/mL and 70 mg/mL of 1-ethyl-3-(3-dimethylaminopropyl)carbodiimide hydrochloride (EDC).

9. The device according to claim 1 for repairing a bone volume of between 2 cm³ and 15 cm³.

10. The device according to claim 1, wherein the three-dimensional mesh comprises filaments with a diameter of 400 µm.

11. The device according to claim 10, wherein the filaments are spaced apart, defining a filament spacing of between 200 µm and 2.5 mm.

12. A method for manufacturing an implantable medical device according to claim 1, wherein the scaffold is manufactured by 3D printing.

13. The manufacturing method according to the claim 12, further comprising a step of sterilising the implantable medical device.

14. A method for bone repair following a loss of bone substance wherein the implantable medical device according to claim 1 is implanted for repairing a bone volume of between 2 cm³ and 15 cm³.
